(19) United States
(12) Patent Application Publication
(10) Pub. No.: US 2010/0316739 A1
(43) Pub. Date: Dec. 16, 2010

(54) Chitosan gel for dermatological use, production method therefor and use of same

(76) Inventor: Ariel Shaul Hasson Nisis, Santiago (CL)

Correspondence Address: HUGHES HUBBARD & REED LLP, ONE BATTERY PARK PLAZA, NEW YORK, NY, 10004, US

(21) Appl. No.: 12/863,551

(22) PCT Filed: Jan. 21, 2009
(86) PCT No.: PCT/IB2009/050228

§ 371 Date: Aug. 31, 2010

(30) Foreign Application Priority Data
Jan. 18, 2008 (CL) 0156-2008

(51) Int. Cl. A61K 36/185; A61P 17/02

(52) U.S. Cl. 424/725

(57) ABSTRACT

Pharmaceutical compositions, based on chitosan, which are suitable to leave a protecting coating on the skin, as well as agents conferring curative, soothing and moisturizing properties to sore skins, are protected.

This gel is characterized by incorporating chitosan in a percentage between 2 and 8% and other components, also in defined proportions such as Portulaca, between 0.1 and 90% and Panthenol in a proportion between 0.1 and 45%, conferring better therapeutic characteristics to the product and other components providing its own characteristics to the gel and conferring it a suitable and long lasting stability.

This gel is for external application and can be employed in sore skins, superficial burns, skin abrasions, post-peeling, post-laser and eroded skins in general.

The present invention relates to a chitosan based gel and more particularly to gels formulated with excipients permitting the gel to lave a protective coating on the skin after the application thereof.

This invention relates to a gel for topical application, characterized by incorporating chitosan as the main component.

Chitosan is from natural origin, therefore it is biocompatible. Furthermore, it has antimicrobial properties (fungicide and bactericide) which are inherent to its intrinsec characteristics. It corresponds to a linear Polysaccharide of deacetylated N-Acetyl-D-Glucosamine and commercially obtained by deacetylation of chitin.

Chitosan has been useful in the absorption of heavy metals in water flows and industrial effluents and also in the development of cosmetic preparations.

Furthermore, partially acetylated chitosan and chitin derivatives have been importantly investigated to seek for therapeutic substances and prosthesis or implant materials. The biocompatibility of materials containing chitosan has been specifically evaluated in blood, wounds and bones.

Chitosan has also been tested in systems for administering pharmaceuticals by means of gels. (Cohya and Cols (1993) J. Microencapsulation, 10(1):1-9).

To carry out the present invention several mixtures have been employed to obtain solutions that allowed to prepare gels in percentages between 2% and 8% of chitosan, in combination with other components conferring therapeutic characteristics suitable for the product, in order to be applied to sore skins, superficial burns, skin abrasions, post-peeling, post-laser and eroded skins in general.

### DESCRIPTION OF THE MATTER KNOWN IN THE ART

Several types of gels have been disclosed for the treatment of superficial burns and sore skins.

Hydrogels, in general, have a high biocompatibility and are good candidates to be employed in medical implants, treatment of wounds and also as controlled and long lasting release matrices for pharmaceuticals.

Degradation in vivo and in vitro of chitin and chitosan has been studied by Tomihata (K. Tomihata and Ikada, Biomaterials 18, 567-575 (1997)). The degradation in vitro can be measured relative to the speed of the lysozyme hydrolysis and the degradation in vivo in studies with testing animals.

The results show that a 100% acetylated chitin and a 69% acetylated chitosan 69%, degrade rapidly.

Several hydrogels for the treatment of wounds and burns have been disclosed, a gel formed by chitosan, glycerol and water, forming a protecting membrane when applied on the skin, is employed in a concentration between 1-4% of chitosan and preferably, a concentration of 1%. This gel is basically recommended as a carrier for pharmaceuticals, such as vasodilators, silver sulfadiazine, analgesics and anti inflammatories (U.S. Pat. No. 4,659,700).

Other hydrogels may acquire insoluble characteristics by the addition in excipients with opposite charge (U.S. Pat. No. 5,620,706).

The hydrogels comprise a poly cation, similar to chitosan, or a polybasic pharmaceutical, that complexes with a poly anion such as Xanthan. It is rather employed in controlled release formulations by encapsulation.

Other gels that have been disclosed are obtained by the interaction of chitosan with polyvinylpyrrolidone, are gels of poly(N-vinyl-lactam)-chitosan and more particularly gels that are absorbent and may adhere to the skin, are flexible and may adapt to a contour. This hydrogel, depending on the other components in the formulation, can be employed for coating wounds, burns, cosmetic masks and cosmetic enamel coatings (ES 2117825).

Other gels have been disclosed, also based on chitosan and polyvinylpyrrolidone, comprising a mixture of neutralized chitosan and a poly(n-vinyl lactam), with and without a plasticizer. The gel may be formed as a wound pack or bandage, wherein the hydrocolloid is capable of absorbing exudates without losing its structure. Also it can be employed as a medicament carrier for transdermal devices (WO2002/02079).

Other gels of chitosan forming part of the prior art, are based on chitosan, between 0.1 and 0.5%, a salt of polyalcohol or a sugar between 1 and 2%. The mentioned salt may be dibasic glycerol monophosphate.

This gel may be formed in situ and may be parenterally administered or also may serve as a vehicle for administration of a pharmaceutical product (ES 2205471).

Other formulations of chitosan disclosed as in gel forms comprise the use of chitosan or a salt thereof, a polyphosphate o sugar phosphate, a plasticizer and a therapeutic agent. It relates to an aqueous solution or suspension wherein the plasticizer is triethyl citrate. The employed chitosan has a deacetylation degree of 40% or higher.

This gel is employed for the administration of medicaments passing through the mucosa, that is, the application is carried out in the noses, vagina, rectus, mouth or eyes (WO2005/079749).

Gels for treating also burns have been disclosed, the gels being based on partially hydrolyzed collagen, with the addition of galactose monomers, pectin and hydroxyethylcellulose, useful in the treatment of first and second degree burns (US2003/0008830).

Other disclosed gels, that are part of the prior art, are formed by mixtures of a hydrophilic poly(aldehyde) and a polymer selected from the group consisting of a poly(amide), a poly(amine), a poly(alcohol) and mixtures thereof, including natural polymers, within the poly(amines) is the chitosan and mixtures thereof (WO00/47149).

This gel is basically employed as a carrier for an active agent or mixtures thereof, also as a gauze for wounds.

Also gels for treating burns based on other polymers not including chitosan, prepared on the base of polyvinyl alcohol, Agar-Agar and one or more natural polymers, such as: gelatine, carrageenan, sodium alginate, carboxymethylcellulose, guar gum and gum acacia (WO01/30407) have been disclosed.

Other chitosan based hydrogels have been disclosed, which are rather employed as antiseptics. It refers to iodate chitosan, prepared on the basis of chitosan, propylene glycol, ethylene glycol, etc. in a concentration range of between 5 and 25% and elemental iodine in a concentration range of between 0.05 and 1% (US2002/0119205).

The human body has an average of a little more than 1.9 m² of skin, which is vital for regulating the temperature of the body in addition to serve as a barrier to diseases and allow the elimination of body wastes and protect the body, among other functions. When the skin is destroyed or damaged by any wound, the traumatic effect on the skin produces not only the physical damage but also the interruption of the physiologic functions of internal tissues.

The treatment of the skin can be done by the use of irrigating solutions and covering sponges also known as porous solids.

Like the chitosan, the chitin has been analyzed because this is similar, in its characteristics, to the glicosamine-glicanes present in the skin, because the repetitive unit of the chitin, n-acetyl glucosamine, is present in the hyaluronic acid and is responsible of the formation of the fibrous net of the proteins, joined during cicatrisation of wounds. Therefore, the chitin compound also has been observed by several investigators as a promoter of a rapid dermal regeneration and accelerator of cicatrisation processes of wounds, since the velocity of cicatrization shown by the chitin has caused the same to be employed as a component product in pharmaceuticals for application in wounds.

The chitosan is, after the chitin, an accelerator of cicatrization process, because of the less effectivity in the capacity of accelerated cicatrization of glucosamine residue in chitosan, as compared to the n-acetylglucosaminate of the chitin.

The chitin is extensively found in the nature, either in the vegetal and animal kingdom. Indeed, it is the second more abundant natural polymer, only exceeded by the cellulose, thus constituting an important renewable resource. This polymer is comprised of amino sugars joined to each other by glycosidic bonds β(1→4) forming a linear chain of N-acetyl-2-amino-2-desoxy-D-glucose units some of which are deacetylated. It is sustained that the natural chitin has a acetylation degree of 66%, that is, one out of three units are deacetylated.

The chitosan is a linear polysaccharide that is obtained by extensive deacetylation of the chitin and is composed of two types of structural units arranged in a random manner (Bernoullian distribution) along the chain, the N-acetyl-D-glucosamine and the D-glucosamine, which are joined to each other by bonds of the type β(1→4) glycosidic bond. In FIG. 1.2 the structure of a totally deacetylated chitosan is shown. However, it is difficult to totally deacetylate the chitin, and what is usually known as chitosan is a family of chitins having different deacetylation degrees, generally over than 45%. The capacity of the chitosan to dissolve in diluted acid aqueous solutions is the commonly accepted criteria to differentiate it from the chitin.

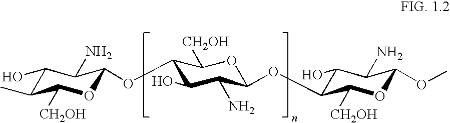

All the bio-materials that are employed in clinical means must have excellent surface properties because they enter in direct contact to live tissue when this material is implanted into the body. The initial response from the body against the bio material depends on such properties.

The chitosan may be used as artificial skin for accelerating cicatrization and in the recovery of ulcers or as a vehicle for drugs releasing. The positive charges of the chitosan and its biocompatibility allows the chitosan to be a support for effective cellular growth.

In addition to the chitosan, natural polymers that confer special properties to the gel formulations, and other components that allow the product to have better effects on the sore skin, burns and superficial wounds, are included.

One of them is the panthenol, also known as pro-vitamin B5. The D-panthenol is converted in the body into vitamin B5, a normal constituent of the skin and the hair, the topical application of same accelerates the cellular regeneration and it is a powerful moistener.

When topically applied, the panthenol is converted into d-pantothenic acid (or simply pantothenic acid) of healthy skin, which actuates in the generation cycle of biochemical energy by the mytocondrya and it is fundamental for the lipid and protein synthesis and the chain bond between lipids-proteins and carbohydrates.

When the amount of d-pantothenic acid is insufficient, well known dermal disorders appear such as keratinization, depigmentation and exfoliation.

The panthenol is rapidly absorbed through the skin, converting into pantothenic acid.

The cellular regeneration is accelerated by the topical application of d-panthenol, since it has a high capacity to penetrate the skin and a characteristic moistening power. Furthermore, it contributes to moisten aged skins and it is ideal for application on light sores in the skin. (Lawrence Sweetman, Encyclopeia of Dietary Supplements, Pantothenic Acid, February 2005).

This chitosan gel may be added also with other bio-compatible substances that provide advantages when administered topically in the treatment of eroded skin. One of these substances is the portulaca, that is obtained from an extract of a vegetal called Portulaca oleracea L. The extract has a good anti sore and anti inflammatory effect when is topically employed. The portulaca extract has been successfully employed in several conditions of the skin, such as: burns, insect bites, pain, inflammations, wounds, ulcers, pruritus, eczema and abscess (Pedro Sánchez Sambucety et al. Las plantas silvestres. Los remedios históricos dermatológicos Med. Cutan Iber. Lat. 2003, 31(5): 337-346). Other natural compounds having curative properties and that also can be employed due to their curative properties are the following extracts Boswellia sacra, Stellaria media, Plantago major, Calendula officinalis, Boswellia carterii, Commiphora myrrha, Chamaemelum nobile, Pelargonium graveolens, and linoleic acid.

### OBJECTIVES

1. Preparation of a chitosan gel (Poly-β-1,4-D-Glucosamine) for use in eroded skins using as a base chitosan and additives conferring the chitosan tissue regenerating properties and soothing properties for the pain present in damaged skins, such as d-panthenol, portulaca extract and other substances having similar characteristics, with biocompatible properties and properties to relieve the erosion symptoms.

2. Preparation of chitosan gels having gaseous permeability properties, to allow a good skin oxygenation, permitting to cure wounds. Also the gel must have the capacity of absorbing the exudates from the surface of the skin and having a middle viscosity, lower than 30,000 cps and sustained in the time. This last characteristic is very important in the application of the product.

It has been surprisingly discovered that the above is achieved with the addition of more than one preservative, that is, for achieving a good stability for the final product the use of mixtures of preservatives are required. The gel must have a high capacity of absorbing water and providing a soothing and moistening action on the damaged skin.

The gels of the invention, due to the incorporation of substances with activity beneficial to treat eroded skins, allow treating superficial wounds of the skin.

3. Another object of the invention was to obtain a gel with tested adherence to the wounds and superficial burns of the skin and smooth action, without causing any risk in the maintenance of a healthy skin.

Another object of the invention was to obtain chitosan gels, formed by dissolution in distilled water, and with the help of an organic acid and the addition of innocuous preservatives and substances that alleviate the wounds of the skin and help in the regeneration of tissues, with excellent properties for recovering the skin damaged by superficial burns or erosions.

The gels of the invention may be employed, either in animals and human beings because they are biocompatible and they are absorbed in the organism preventing the same from infections.

The gels of the invention remain stable in the time and keep their capacity to adhere to the skin at room temperature. Mostly, their stability is provided by the preservatives, which are essentials because of their type of formulation and application.

The preservatives must keep, during all the life of the product, the absence of pathogenic microorganisms and also keep the essential characteristics of the formulation, such as the pH and the viscosity, what is surprisingly achieved only by mixtures of preservatives which, between the more suitable, are: Methylparaben, Propylparaben, Butylparaben, Diazolidinyl urea, Imidazolidinyl urea, Phenoxyethanol, DMDM Hydantoin, Sodium Hidroxymethylglicinate, Iodine propinyl, Butylcarbamate, Chloromethyl, Isotiazolinona, Methylisotiazolinona, Hidroxyacetic Acid, Bencilic Alcohol, Benzethonium Chloride, Chlorphenesin, Benzoic Acid, Butylenglycol, Sorbic Acid, Polyaminopropylbiguanide, Gluconolactone, Sodium Benzoate and Calcium Gluconate.

The gel is kept transparent and allows to permanently observe the evolution of the wound, it may be employed as ion retention membranes and/or as skin protector, either in animals and humans, because it helps restore skin and tissues which are burn, ulcerated or damaged.

The gels must be easy to apply on the skin, flexible and transparent with smooth adhesivity and easy to remove. They may be employed as growth support, cellular proliferation and differentiation (fibroblasts and keratinocytes); it may be obtained in gel films having different thicknesses, depending on the lesion and the object of the application in the wound that is treated with the gel (20-50 um).

Furthermore, due to the type of application, the gels must have hypoallergenic characteristics.

### DESCRIPTION OF THE INVENTION

Chitosan (poly-β-1,4-D-glucosamine) is a derivative of a natural polysaccharide, known under the name of chitin (poly-β-1,4-D-acetylglucosamine) that is mainly found in the exoskeleton of crustaceous shells.

The properties of the biodegradable polymers may be modified and enhanced by the addition of several substances (chitosan, portulaca, alpha hydroxy acid panthenol and innocuous preservatives). By using components having well known biodegradability and therapeutic properties in burns and eroded and damaged skins in general, the gels supported by the invention are entirely biodegradable and with improved properties, and can be obtained at low costs.

The chitosan gels are obtained by dissolution of chitosan in organic acid solutions, mainly of the type alpha-hydroxy acids, such as: glycolic acid, lactic acid, alpha hydroxyethanoic acid, alpha hydroxyoctanoic acid, alpha hydroxycaprilic acid, malic acid, citric acid and tartaric acid, having good solubility in water. The hydrogels are obtained by using chitosan having different molecular weights between 25,000 and 1,250,000 g/mol and with a deacetylation degree higher than 80%.

The properties of the hydrogel are improved by the addition of panthenol and portulaca or another natural substance having similar characteristics, each one in a proportion varying between 0.1 and 45%.

Surprisingly, a stable preparation at a pH between 5.0 and 5.9 has been prepared with chitosan as the only one viscosifier in a percentage between 0.2 and 10%, with the addition of at least two preservatives, also including portulaca and panthenol or another natural component, which confer relevant features for the treatment of burns type or erosion type lesions of the skin, thus achieving a rapid regeneration and cicatrization of the damaged tissues.

In the sequence of pictures appearing below, the effect of the hydrogel on the damaged skin is appreciated.

FIGS. 2 and 3 show the area of the leg alter the application of the hydrogel, with a coating of the protective and tissue regenerator product.

Methodology of Preparation of the Hydrogel

In a stainless steel container, having distilled water, the diluted organic acid is added, it is homogenized and the pH is kept between 4.0 and 4.5, during the manufacturation of the hydrogel; after that all the chitosan, as appearing in the formulation, is slowly added under continuous stirring up to complete the dissolution.

Subsequently, the preservatives polyaminopropyl biguanide, Gluconolactone, Sodium Benzoate and Calcium Gluconate or mixtures of the other preservatives which are included in this patent, are added; subsequently the portulaca or another natural extract is added and, after that the panthenol is added.

Finally the pH is adjusted between 5.0 and 5.9 by employing an organic acid solution diluted between 1% and 2% or a 2% sodium hydroxide solution

Subsequently, the preparation is stored in a container until fractioning.

The compositions are indicated in this patent as examples and they do not restrict the scope and protection of the invention.

### EXAMPLE 1

| Chitosan Powder | 3.50 | g |
|---|---|---|
| Polyaminopropyl biguanide | 1.50 | g |
| Portulaca oleracea Extract | 4.00 | g |
| Panthenol | 4.00 | g |
| Citric acid 1% w/v q.s. | pH 5.5 | |
| Distilled water q.s. | 100 | g |

### EXAMPLE 2

| Chitosan Powder | 4.0 | g |
|---|---|---|
| Polyaminopropyl biguanide | 1.5 | g |
| Portulaca oleracea Extract | 6.0 | g |
| Panthenol | 4.0 | g |
| Citric acid 1% w/v q.s. | pH 5.5 | |
| Distilled water q.s. | 100 | g |

### EXAMPLE 3

| Chitosan Powder | 4.0 | g |
|---|---|---|
| Polyaminopropyl biguanide | 1.5 | g |
| Portulaca oleracea Extract | 4.0 | g |
| Panthenol | 6.0 | g |
| Lactic acid 1% w/v q.s. | pH 5.5 | |
| Distilled water q.s. | 100 | g |

### EXAMPLE 4

| Chitosan Powder | 8.0 | g |
|---|---|---|
| Polyaminopropyl biguanide | 1.5 | g |
| Portulaca oleracea Extract | 4.0 | g |
| Panthenol | 6.0 | g |
| Citric acid 1% w/v q.s. | pH 5.5 | |
| Distilled water q.s. | 100 | g |

### EXAMPLE 5

| Chitosan Powder | 3.50 | g |
|---|---|---|
| Polyaminopropyl biguanide | 1.50 | g |
| Gluconolactone/Sodium Benzoate/Ca⁺⁺ Gluconate | 1.50 | g |
| Portulaca oleracea Extract | 4.00 | g |
| Panthenol | 4.00 | g |
| Lactic acid 1% w/v q.s. | pH 5.5 | |
| Distilled water q.s. | 100 | g |

### EXAMPLE 6

| Chitosan Powder | 4.0 | g |
|---|---|---|
| Polyaminopropyl biguanide | 1.50 | g |
| Gluconolactone/Sodium Benzoate/Ca⁺⁺ Gluconate | 1.50 | g |
| Portulaca oleracea Extract | 6.00 | g |
| Panthenol | 4.00 | g |
| Lactic acid 1% w/v q.s. | pH 5.5 | |
| Distilled water q.s. | 100 | g |

### EXAMPLE 7

| Chitosan Powder | 4.0 | g |
|---|---|---|
| Polyaminopropyl biguanide | 1.50 | g |
| Gluconolactone/Sodium Benzoate/Ca⁺⁺ Gluconate | 1.50 | g |
| Portulaca oleracea Extract | 4.00 | g |
| Panthenol | 6.00 | g |
| Lactic acid 1% w/v q.s. | pH 5.5 | |
| Distilled water q.s. | 100 | g |

### EXAMPLE 8

| Chitosan Powder | 8.0 | g |
|---|---|---|
| Polyaminopropyl biguanide | 1.50 | g |
| Gluconolactone/Sodium Benzoate/Ca⁺⁺ Gluconate | 1.50 | g |
| Portulaca oleracea Extract | 4.00 | g |
| Panthenol | 6.00 | g |
| Lactic acid 1% w/v q.s. | pH 5.5 | |
| Distilled water q.s. | 100 | g |

TABLE 1
Stability of the compositions

| | MONTH 1 | | | MONTH 2 | | | | MONTH 3 | | | |
|---|---|---|---|---|---|---|---|---|---|---|---|
| Examples | viscosity | pH | Microbiology | viscosity | | pH | Microbiology | viscosity | | pH | Microbiology |
| 1 | 10,000 cps | 5.5 | lower than | 100 | cps | 5.4 | higher than | 100 | cps | 5.4 | higher than |
| | | | 100 | | | | 1000 | | | | 1000 |
| 2 | 12,000 cps | 5.5 | lower than | 50 | cps | 5.5 | higher than | 50 | cps | 5.5 | higher than |
| | | | 100 | | | | 1000 | | | | 1000 |
| 3 | 12,000 cps | 5.5 | lower than | 200 | cps | 5.4 | higher than | 200 | cps | 5.2 | higher than |
| | | | 100 | | | | 1000 | | | | 1000 |
| 4 | 15,000 cps | 5.5 | lower than | 200 | cps | 5.3 | higher than | 200 | cps | 5.3 | higher than |
| | | | 100 | | | | 1000 | | | | 1000 |
| 5 | 11,000 cps | 5.5 | lower than | 11,500 | cps | 5.5 | lower than | 11,900 | cps | 5.5 | lower than |
| | | | 100 | | | | 100 | | | | 100 |
| 6 | 14,000 cps | 5.5 | lower than | 14,800 | cps | 5.4 | lower than | 14,600 | cps | 5.4 | lower than |
| | | | 100 | | | | 100 | | | | 100 |
| 7 | 15,000 cps | 5.5 | lower than | 15,300 | cps | 5.4 | lower than | 15,100 | cps | 5.5 | lower than |
| | | | 100 | | | | 100 | | | | 100 |
| 8 | 18,000 cps | 5.5 | lower than | 18,200 | cps | 5.5 | lower than | 18,100 | cps | 5.5 | lower than |
| | | | 100 | | | | 100 | | | | 100 |

Specifications of the final product:

Aspect, gel having a middle viscosity, free of strange particles in suspension, of a light yellow color and traslucid. Viscosity, values between 5,000 and 20,000 cps.

pH values between 5.0 and 5.9

Microbiologic: no mesophilic aerobic germs and pathogens, ≦100 UFC/gram and Fungus and Yeast ≦100 UFC/g;

In table 1 the importance of including the hydrogels of the preservatives mixture in the compositions may be observed, in examples 1 to 4 the compositions have lower variations, but the most important is that only one preservative is included, in examples 5 to 8, the compositions have lower variations, but the most significant is the inclusion within the components of a mixture of preservatives. From the results of the table it may be appreciated that, at the second month of the study of a significant change in the viscosity, a drastic decrease is produced in the formulations 1 to 4 as well as an evident microbiological contamination, specifically by fungi. This has not occurred in the formulations of Examples 5 to 8, which include the mixture of preservatives.

This experimental text has been carried out under normal room conditions for temperature and relative humidity (25°±2° C. and 60 ±5%H).

It is not usual and it is surprising the fact that it was necessary to use a mixture of preservatives for preserving the conditions (specifications of the product), because it would have been expected that the use of one preservative would be enough to keep the stability of the product. It was not foresaw that the use of only one preservative would result in the variations observed for the viscosity.

1. A stable hydrophilic based polymer gel for topical application, characterized in that:
a) It contains chitosan,
b) The chitosan is dissolved by the addition of a solvent,
c) It contains one or more substances that increases the capacity of tissue restoration.
d) It contains preservatives that are innocuous to the skin.

2. A gel according to claim 1, characterized in that the chitosan that has a molecular weight between 25,000 and 1,250,000 g/mol., is the main constituent of the hydrogel and it is present in proportions varying between 2 and 8%.

3. A gel according to claims 1 to 2, characterized in that the solvent is a diluted organic acid, from the alpha hydroxyl acids family, such as: glycolic acid, lactic acid, alpha hydroxyethanoic acid, alpha hydroxyoctanoic acid, alpha hydroxycaprilic acid, malic acid, citric acid and tartaric acid.

4. A gel according to claims 1 to 3, characterized in that the substances of natural origin, that enhances the tissue restoration capacity, are selected from Portulaca oleracea extract, Boswellia sacra extract, Stellaria media extract, Plantago major extract, Calendula officinalis extract, Boswellia carterii extract, Commiphora myrrha extract, Chamaemelum nobile extract, Pelargonium graveolens extract, panthenol and linoleic acid.

5. A gel according to claims 1 to 4, characterized in that the substance(s) of natural origin are present in a percentage varying between 0.1 and 45%.

6. A gel according to claims 1 to 5, characterized by including a mixture of preservatives each one in a concentration varying between 0.1 and 30%.

7. A gel according to claim 6, characterized in that the preferred preservative is a mixture of polyaminopropyl Biguanide, Gluconolactone, Sodium Benzoate and Calcium Gluconate.

8. Method for obtaining the gel for topical use of claims 1 to 7, characterized by the following steps:
a) Preparing the solutions of chitosan, from a 1-2%, w/v solution of a weak organic acid containing between 2 and 8% w/w of chitosan.
b) Adding to the solution obtained in (a) the mixture of preservatives in a proportion varying between 0.1 and 30%.
c) Then, once the mixture is homogeneous, adding under continuous stirring, the substances of natural origin in a proportion varying between 0.1 and 45%.
d) Finally, adjusting the pH between 5.0 and 5.9 with a 2% sodium hydroxide solution or an organic acid solution diluted between 1 and 2%.

9. Method for obtaining an hydrochitosan gel, according to claim 8, characterized in that, as organic acid, an alpha hydroxy acid is employed, such as: glycolic acid, lactic acid, alpha hydroxyethanoic acid, alpha hydroxyoctanoic acid, alpha hydroxycaprilic acid, malic acid , citric acid and tartaric acid.

10. Use of the gel of claims 1 to 7, characterized in that it is for the treatment of sore skins and burns by topical application in thin layers.

11. Use of the gel of claims 1 to 7, characterized in that it is for the treatment of dermal abrasions, in post peeling and post laser by application in thin layers on the affected skin.

12. Use of the gel of claims 1 to 7, characterized in that it is for the treatment of eroded skins by application in layers on the surface under treatment.
